(19) United States
(12) Patent Application Publication
(10) Pub. No.: US 2025/0147014 A1
(43) Pub. Date: May 8, 2025

(54) PLASMON-ENHANCED FLUORESCENCE BIOCHEMICAL SENSORS

(71) Applicant: The Regents of the University of Colorado, a body Corporate, Denver, CO (US)

(72) Inventors: Anatoli Pinchuk, Colorado Springs, CO (US); Tetyana Sergeyeva, Kyiv (UA); Daria Yarynka, Kyiv (UA); Vladimir Chegel, Kyiv (UA); Andri Lopatynskyi, Kyiv (UA)

(73) Assignee: The Regents of the University of Colorado, a body, Denver, CO (US)

(21) Appl. No.: 18/837,842

(22) PCT Filed: Feb. 14, 2023
(86) PCT No.: PCT/US23/13033

§ 371 Date: Aug. 12, 2024

Related U.S. Application Data
(60) Provisional application No. 63/267,996, filed on Feb. 14, 2022.

(51) Int. Cl. G01N 33/543; G01N 21/64; G01N 33/53

(52) U.S. Cl. CPC G01N 33/54373; G01N 21/645; G01N 33/5308; G01N 33/54346; G01N 2021/6463; G01N 2333/37; G01N 2600/00

(57) ABSTRACT

A highly sensitive technology enabling selective recognition of aflatoxins such as in contaminated cereal samples. Methods for the synthesis of AgNP-containing MIP membranes from the monomer mixtures containing both methacrylate monomers and oligomers as well as AgNOs solution in dimethylformamide enable the detection system. The AgNP were formed in the structure of the polymeric membranes during the pre-heating step followed by the UV-initiated polymerization. The highly selective sensor elements where MIP membranes are combined with the Ag nanostructures as signal amplifiers (LSPR-MIP nanochips) can be stored separately and used with a simple optical recognition system as selective elements of point-of-care sensor devices, even in in-field conditions. Composite plasmonic membranes provide both highly selective recognition of the target toxin and the generation of an enhanced optical signal, allowing aflatoxin B1 detection at ultralow concentrations.

### CROSS-REFERENCE TO RELATED APPLICATIONS

This application claims the benefit of U.S. Provisional Application No. 63/267,996, filed on Feb. 14, 2022.

### STATEMENT OF GOVERNMENT INTEREST

This invention was made with government support under grant number 65495, awarded by the U.S. Civilian Research & Development Foundation (CRDF Global). The government has certain rights in the invention.

### FIELD OF INVENTION

This invention relates to biochemical sensors. More particularly, this invention relates to methods and devices for a sensor platform using plasmon-enhanced fluorescence biochemical sensors with enhanced sensitivity and selectivity for the detection of analyte molecules such as food toxins.

### BACKGROUND OF THE INVENTION

There is an urgent need for rapid, low-cost, easy-to-use, and highly sensitive biochemical sensors. [Mitchell N J, et al., Food Addit Contam Part A Chem Anal Control Expo Risk Assess. 2016; 33(3):540-550.] Fluorescence-based detection is a promising approach in biosensor technology. It provides rapid and sensitive detection of analyte molecules, such as mycotoxins. A number of fluorescent sensors based on natural enzymes and receptors have been developed. [Campos, W. E. O. et al., (2017) Journal of Food Composition and Analysis, 60: 90-96; Ivešić, M., et al., (2018) Toxicology Letters, 295(1): S147-S148; Zhan, S. et al., (2021) Food Chemistry, 342: 128327; Wu, J., et al., (2019) Food Chemistry, 298: 125034; Mousivand, M. et al., (2020) Analytica Chimica Acta, 1105: 178-186; Tang, L., et al., (2020) Talanta, 214:

The use of nanomaterials provides another promising approach in biosensor technology. Specifically, the application of a wide range of nanomaterials has attracted interest for the use in biosensors due to their unique optical characteristics. For example, noble metal nanoparticles can modify spontaneous emission of nearby fluorescent molecules and improve the limit of detection of the analyte of interest. [Sharma, A., et al., (2018) Toxins. 10(5): 197; Tam, F., et al., (2007) Nano Letters, 7(2): 496-501] The present invention meets the needs for a rapid, low-cost, easy-to-use, and highly sensitive biochemical sensor through the provision of a sensor platform using plasmon-enhanced fluorescence biochemical sensors using a combination of fluorescence-based detection coupled with nanomaterials.

### SUMMARY OF THE INVENTION

The present invention provides a novel sensor platform with enhanced sensitivity and selectivity for the detection of analyte molecules, such as food toxins. The approach is based on a molecular imprinted polymer (MIP) membrane that provides selective binding of the analyte molecule (e.g., aflatoxin B1) and plasmon-enhanced fluorescence from the analyte molecule by a local electric field generated in close proximity to silver nanoparticles (or other noble-metal nanoparticle, such as AuNP) excited at the surface plasmon resonance wavelength. Molecularly imprinted polymers (MIPs) with supramolecular aflatoxin-selective receptor sites and embedded silver nanoparticles were prepared as thin (e.g., 1-1,000 micrometers) polymer films immobilized on the surface of a glass slide using in situ polymerization. Polymer substrates could be used in place of the glass slide. The detection limit of the sensor for aflatoxin B1 is 0.3 ng mL⁻¹, which is significantly lower compared to a fluorescent sensor without silver nanoparticles. The plasmon-enhanced fluorescence factor is 33. The linear dynamic range of the sensor was estimated as 0.3-25 ng mL⁻¹.

Plasmon-enhanced fluorescence combined with the molecular-imprinted polymer membranes, or thin films, with incorporated plasmonic nanoparticles can serve as an alternative to natural antibodies and receptors for the fluorescent affinity sensor and facilitate rapid and sensitive detection.

In a first aspect the present invention provides a sensor chip having an MIP membrane or thin film where the MIP membrane or thin film has embedded silver nanoparticles (AgNP). The MIP or thin film is constructed to exhibits a binding affinity for a fluorophore of interest. In other words, the MIP exhibits specificity in binding for the particular fluorophore. The silver nanoparticles in the MIP enhance the fluorescence of the fluorophore upon application of UV irradiation. Thus, the fluorescence can be more readily detected.

In an advantageous embodiment, the MIP of the first aspect selectively binds a fluorophore selected from the group consisting of aflatoxin B1 (AFB1), aflatoxin B2 (AFB2), aflatoxin G1 (AFG1), aflatoxin G2 (AFG2), deoxynivalenol (DON), zearalenone (ZEA), fumonisin B1 (FB1), fumonisin B2 (FB2), fumonisin B3 (FB3), ochratoxin (OhA), and trichothecene.

The fluorophore of interest can be a mycotoxin. Advantageously it is an aflatoxin. In particularly advantageous embodiments the aflatoxin is aflatoxin B₁, aflatoxin B₂, aflatoxin G₁, or aflatoxin G₂.

In further embodiments according to the first aspect the AgNP are roughly spherical in shape and have a size of about 30-70 nm. The AgNP are nanoparticles can be evenly distributed in the structure of the MIP membrane to achieve consistent results.

The MIP can be made from a polymer such as acrylamide (AA), poly(ethyleneimine), poly(hydroxyethyl methacrylate), poly(vinylpyrrolidone), novolak, poly(4-vinylphenol), poly(4-vinylphenol)-co-(methyl methacrylate), and poly(styrene-co-allyl alcohol).

The sensor chip can have an MIP with an aptamer that selectively binds a fluorophore of interest. In an advantageous embodiment the aptamer that selectively binds an aflatoxin.

In a second aspect the present invention provides a sensor chip sensor chip constructed of an MIP membrane or thin film having embedded nanoparticles where the nanoparticles are silver nanoparticles, gold nanoparticles, copper nanoparticles or aluminum nanoparticles. The MIP or thin film exhibits a binding affinity for a fluorophore of interest, such as an aflatoxin. The nanoparticle in the MIP enhances the fluorescence of the fluorophore upon application of UV irradiation.

In a third aspect the present invention provides a method of detecting a fluorophore in a sample. The method includes the steps of providing a sensor chip such as one of the sensor chips from the first two aspects, above, contacting the sensor chip with a sample to be tested for the presence of a fluorophore under conditions effective to cause binding between fluorophores in the sample and the sensor chip, irradiating the contacted sensor chip with UV light, and detecting the resulting fluorescence in the sample. The resulting fluorescence is enhanced by the AgNP nanoparticle in the sensor chip. If the detected fluorescence exceeds a threshold level then the fluorophore is deemed to be present in the sample. In an advantageous embodiment the fluorophore of interest is an aflatoxin. In further advantageous embodiments the nanoparticle is AgNP. The concentration of fluorophore in the sample can be quantified by comparing the detected fluorescence in the sample with a standard curve of fluorescence for the concentration of the fluorophore. In an advantageous embodiment of the method of detecting a fluorophore in a sample the excitation wavelength is about 365-400 nm and the measurement range is about 395-550 nm. In a particularly advantageous embodiment the excitation wavelength is about 365 nm.

In a fourth aspect the present invention provides the optical setup for a portable fluorimeter. The optical setup includes two collimators, a bandpass filter (e.g., 365 nm), a sample holder, a nanochip with high-conductive nanoparticles as in one of the first two aspects, above, and a longpass filter (400 nm).

In a fifth aspect the present invention provides a method for the synthesis of AgNP-containing MIP membranes from monomer mixtures. The method according to the fifth aspect includes the steps of providing a solution containing methacrylate monomers or oligomers and AgNO₃ in dimethylformamide, forming the AgNP in the structure of the polymeric membranes during the pre-heating step and polymerzing the solution using UV-initiated polymerization. The concentration of AgNO3 in the solution can be between 1.0 and 2.0 mM. Advantageously, the concentration of AgNO3 is about 1.5 mM.

In a sixth aspect the present invention provides a sensor platform comprising a plasmon-enhanced fluorescence biochemical sensor. The sensor has enhanced sensitivity and selectivity for the detection of analyte fluorophore molecules such as food toxins relative to a sensor not having plasmon-enhanced fluorescence.

In a seventh aspect the present invention provides a method for the synthesis of AgNP-containing MIP membranes from the monomer mixtures containing both methacrylate monomers and oligomers as well as AgNO₃ solution in dimethylformamide, where the AgNP were formed in the structure of the polymeric membranes during the pre-heating step followed by the UV-initiated polymerization. The methodology taught herein creates highly selective sensor elements where MIP membranes are combined with the Ag nanostructures as signal amplifiers (LSPR-MIP nanochips) that can be stored separately and used with a simple optical recognition system as selective elements of point-of-care sensor devices, even in in-field conditions. The novel composite plasmonic membranes provide both highly selective recognition of the target toxin and the generation of an enhanced optical signal, allowing aflatoxin B1 detection at ultralow concentrations.

A highly selective and ultrasensitive surface employing plasmon-enhanced fluorescence can be used for a biochemical portable sensor for rapid detection of analyte molecules such as toxins, chemical biomarkers, etc. where the analyte molecule exhibits some level of fluorescence.

Applications for the technology include food and beverages quality screening, pharmacy and pharmaceutical applications, and a wide array of applications and devices with the medical industry.

### BRIEF DESCRIPTION OF THE DRAWINGS

For a fuller understanding of the invention, reference should be made to the following detailed description, taken in connection with the accompanying drawings, in which:

FIG. 1 is a schematic of the optical setup of portable fluorimeter: elements 1 and 6—collimators, 2—bandpass filter (365 nm), 3—sample holder, 4—nanochip with high-conductive nanoparticles, 5—longpass filter (400 nm).

FIG. 2 is a graph showing a typical calibration curve of the MIP-based fluorescent sensor system for AFB1 detection. Fluorescence of MIP and blank thin films immobilized on the glass surfaces synthesized using acrylamide (AA) as a functional monomer was registered after incubation in 0.3-500 ng mL⁻¹ AFB1 solutions in 20 mM Na-phosphate buffer pH 6.0, containing 10% acetonitrile.

FIG. 3 is a graph showing a typical calibration plot of the fluorescent sensor system based on acrylamide-containing MIP thin film immobilized on the surface of a glass slide for AFB1 detection obtained after incubation of the sensor chips in 5-250 ng mL⁻¹ solutions of AFB1 in 20 mM Na-phosphate buffer pH 6.0, containing 10% acetonitrile.

FIG. 4 is a graph showing a cross-reactivity of differential sensor responses of the fluorescent sensor system based on AFB1-selective MIP thin film immobilized on the surface of a glass slide. Differential sensor responses after addition of 100 ng mL⁻¹ AFB1, AFB2 and OhA (ochratoxin) were estimated. The measurements were performed in 20 mM Na-phosphate buffer pH 6.0, containing 10% acetonitrile.

FIG. 5 is two TEM images labeled (a) and (b) of AgNPs formed in the structure of MIP thin polymer films synthesized with AA as a functional monomer taken at two different magnifications.

FIG. 6 is a graph showing florescent sensor responses of the sensors based on AgNP-containing MIP thin films synthesized with different AgNO₃ concentrations in response to addition of 100 ng mL⁻¹ AFB1. The measurements were performed in 20 mM Na-phosphate buffer pH 6.0, containing 10% acetonitrile.

FIG. 7 is a graph showing the fluorescence spectra of aflatoxin-B1-sensitive and blank membranes obtained in the presence and absence of AgNO₃ in the initial mixture of monomers for the membrane synthesis (100 ng mL⁻¹ AFB1 were added to the analyzed sample): AM—MIP membrane obtained from the monomer mixture containing 1.5 mM AgNO₃; M—MIP membrane obtained from the monomer mixture without AgNO₃; B—Blank membrane obtained from the monomer mixture without AgNO₃; AB—Blank membrane obtained from the monomer mixture containing 1.5 mM AgNO₃. All measurements were carried out using Fluoromax_PLUS_PR928P spectrofluorimeter, excitation wavelength 365 nm, measurement range 395-550 nm.

FIG. 8 is a graph showing typical calibration plots of the AgNPs-containing MIP-membrane-based fluorescent sensor systems for AFB1 detection. Fluorescence of AgNPs-containing MIP and blank thin polymeric films immobilized on the glass slides synthesized with AA as a functional monomer with addition of 1.5 mM AgNO₃. The measurements were performed in 20 mM Na-phosphate buffer pH 6.0, containing 10% acetonitrile.

FIG. 9 is a graph showing a comparison of the values of fluorescent sensor responses obtained from the sensors based on AgNP-containing and AgNP-free MIP chips after addition of 0.1-100 ng mL⁻¹ AFB1. AgNPs-containing MIP thin films were synthesized using AA as a functional monomer with addition 1.5 mM AgNO₃. The measurements were performed in 20 mM Na-phosphate buffer pH 6.0, containing 10% acetonitrile.

FIG. 10 is a graph showing the cross-reactivity of differential sensor responses of the fluorescent sensor system based on AgNPs-containing MIP thin films synthesized with 1.5 mM AgNO₃ and AA as a functional monomer. Differential sensor responses were registered after addition of 15 ng mL⁻¹ AFB1, AFB2, AFG2 and OTA. The measurements were performed in 20 mM Na-phosphate buffer pH 6.0, containing 10% acetonitrile.

### DETAILED DESCRIPTION OF THE PREFERRED EMBODIMENT

Plasmonic nanoparticles can increase quantum yield and improve photostability of fluorophores at specific optimized distance of around 5-90 nm. [Ribeiro, T., et al., (2017) Sci. Rep., 7: 2440; Li, M., et al., (2015) Analyst 140(2): 386-406; Zenin, V. A., et al. (2015) Nano Lett., 15(12): 8148-8154.] This is known as metal-enhanced fluorescence (MEF), or plasmon-enhanced fluorescence (PEF). [Li, M., et al., (2015) Analyst 140 (2): 386-406; Jeong, Y., et al., (2018) Biosensors and Bioelectronics, 111: 102-116.] An enhanced localized electrical field of the nanoparticle at the wavelength of localized surface plasmon resonance (LSPR) results in modified optical characteristics of the nearby fluorophore. Furthermore, plasmonic nanoparticles act as antennas for the fluorophore, that typically results in higher emission intensity. The shape and size of the metal nanostructures are critical for the metal-enhanced fluorescence. Non-radiative energy transfer between the metal nanoparticle and the fluorophore that depends on spectral overlap between the metal surface and the fluorophore plays a crucial role in the fluorescence enhancement. This process is explained by Förster resonance energy transfer (FRET).

Plasmon-enhanced fluorescence (PEF) can be used to increase the sensitivity of fluorescent sensors. PEF provides ten-fold to hundreds-fold enhancement factor and enables the fluorometric registration of various analytes with an attomolar limit of detection as well as their single molecules by exploiting the localized surface plasmon excitation in high-conductive metal nanoparticles.

MIP membranes or thin films with incorporated metal nanoparticles can serve as an alternative to natural antibodies and receptors for the producing sensing chips for the fluorescent affinity sensor. MIPs are generally recognized to be as sensitive and selective as natural receptors, while they normally demonstrate superior stability and inexpensive user-friendly production procedures. On the other hand, AgNP can be used as an enhancer florescence detection and a sensor signal amplifier. Gold and silver nanoparticles can be formed during the in situ synthesis in the structure of a number of polymers, including poly(ethyleneimine), poly(hydroxyethyl methacrylate), poly(vinylpyrrolidone), novolak, poly(4-vinylphenol), poly(4-vinylphenol)-co-(methyl methacrylate), poly(styrene-co-allyl alcohol), and MIPs. [Abargues, R., et al., (2009) New J. Chem, 33(8): 1720; Abargues, R., et al., (2008) Nanotechnology, 19(35): 355308; Aznar-Gadea, E. et al., (2021) ACS Applied Polymer Materials, 3(6): 2960-2970; Riskin, M., et al., (2011) Analytical Chemistry, 83(8): 3082-3088.] The size and shape of the described nanoparticles as well as distance between them, which are the important parameters determining their ability to enhance fluorescence intensity, can be controlled.

Here we introduce novel nanostructured composite polymeric membranes with embedded in situ-synthesized Ag nanoparticles and a portable sensor device prototype for the PEF-based MIP-assisted detection of analyte molecules, such as toxins, chemical biomarkers, etc. The proposed portable biochemical sensor can be used for rapid and reliable identification of toxins and chemical biomarkers.

### Example 1—Materials and Methods

Aflatoxin B1 (AFB1), aflatoxin G2 (AFG2), AgNO₃, ochratoxin A (OTA), acrylamide (AA), dimethylformamide (DMF), polyethyleneglycol Mw 20,000 (PEG 20,000), triethyleneglycoldimethacrylate were purchased from Sigma-Aldrich (St. Louis, USA). Oligourethaneacrylate (OUA) was synthesized as previously described and DMF was distilled under reduced pressure over CaO and P₂O₅. [Yu. L. Spirin, et al., (1968) Vysokomol. Soyedin. A. 8: 2116-2121.] Methacryloxypropyltrimethoxysilane was purchased from Serva (Heidelberg, Germany). Microscope glass slides 25.4×76.2 mm, 1 mm-thick (Marienfeld, Germany) were cut to 13×25.4 mm pieces prior to MIP membranes' immobilization on the slide.

All the other reagents of analytical grade were purchased from Sigma-Aldrich (USA), UkrOrgSyntez (Ukraine), and Macrochim (Ukraine) and used without additional purification. The samples naturally contaminated with aflatoxin AFB1 and characterized by using traditional analytical methods (HPLC and ELISA) were obtained from Romer Labs (Kyiv, Ukraine).

### Example 2—Immobilization of MIP Membranes on the Glass Surfaces

The aflatoxin B1-selective MIP membranes were immobilized on the surface of glass slides treated with γ-methacryloxypropyltrimethoxysilane to provide covalent immobilization of the MIP thin film on the glass surface. The monomer mixture (Table 1, below) was polymerized between two glass slides, one of which was treated with γ methacryloxypropyltrimethoxysilane. The monomer mixtures were heated at 80° C. for 2 min to dissolve PEG 20,000. This was followed by addition of the initiator and a final 30 min-polymerization step initiated by UV light (λ=365 nm, intensity 3.4 W m⁻²). To obtain AFB1-selective MIP membranes with AgNP embedded in their structure, 0.6-2.9 mM AgNO₃ were added to the initial monomer mixtures for the AFB1-selective membranes' synthesis. AgNP were formed in situ during the pre-heating step and further UV-initiated polymerization procedure.

The MIP membranes were synthesized according to a dummy template-based approach with ethyl-2-oxocyclopentanecarboxylate as a template molecule and acrylamide as a functional monomer. [Sergeyeva, T., et al., (2019) Talanta, 201: 204-210.] The corresponding blank membranes were obtained in the absence of ethyl-2-oxocyclopentanecarboxylate so that no aflatoxin B1-selective sites were formed in their structure. The polymerization was followed by 8-h Soxhlet extraction procedure in ethanol for removal of the dummy template and the other monomers, which were not included in the structure of the molecularly-imprinted and blank polymers. That was followed by the other 8-h extraction procedure in distilled water at 80° C. to remove PEG 20,000 responsible for the pore formation in the polymers. Finally, the fully-formed MIP and blank membranes immobilized on the glass slides were dried and stored at room temperature for the further investigations. The thickness of the MIP membranes immobilized on glass surfaces was estimated using Electronic Digital Caliper 0-300 micrometer (“Adoric”, Ningbo, China). It is contemplated that other mechanisms can be used to make an MIP membrane that is selective for a target molecule if interest. For example, aptamers can be used to allow for highly specific binding. One key advantage of using the membranes taught herein is that they remain functional for a much longer time than antibody-based approaches.

### On-Chip Fluorescent Sensor for the Selective Detection of AFB1.

The detection of aflatoxin B1 is based on its natural ability to fluorescence. The sensor signals were generated by UV-irradiation (λ=365 nm) after incubation of both MIP and blank sensor chips in 0.1-500 ng mL⁻¹ AFB1 in 20 mM Na-phosphate buffer pH 6.0, containing 10% acetonitrile. Blue fluorescence of AFB1 selectively adsorbed on the surface of glass chips coated with thin MIP membranes was registered with a standard laboratory spectrofluorometer Fluoromax_PLUS_PR928P (Horiba, Japan). After incubation the MIP and blank membranes were rinsed in 20 mM Na-phosphate buffer pH 6.0, containing 10% acetonitrile and dried at room temperature. The sensor responses were registered with UV-irradiation of the sensor chips (MIP membranes covalently attached to the surface of glass slides) fixed in a membrane holder of spectrofluorometer (Ex bandwidth 5 nm, Em bandwidth 2.5 nm, excitation wavelength 365 nm, measurement range 395-550 nm, and emission wavelength 420 nm) directly at the membranes' surface.

### Portable Fluorimeter for Aflatoxin B1 Detection in In-Field Conditions.

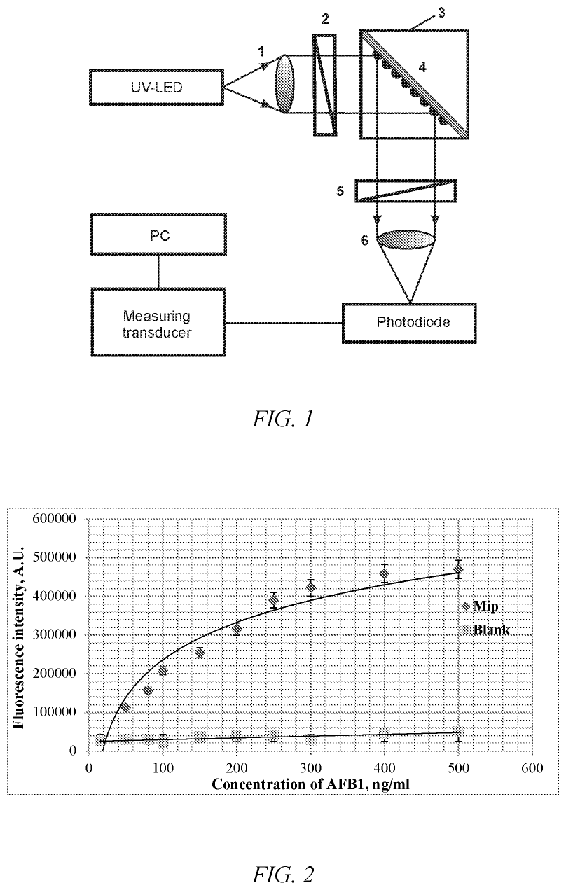

Fluorescence measurements were performed using the Fluoromax_PLUS_PR928P (Horiba, Japan). The fluorescence measurements were also carried out by using the developed portable fluorimeter with the optical signal in the vicinity of wavelength of aflatoxin fluorescence (365-400 nm). The optical scheme of the device is shown in FIG. 1.

The portable fluorimeter uses white or ultraviolet LED as a radiation source, the light flux of which is focused by a collimator and sent through an optical cable to the sample holder with built-in bandpass filter (365 nm). The fluorescent signal amplified by the plasmonic nanochip is collected by a collimator and is registered with photodiode through longpass filter (400 nm).

The recorded signal is transmitted via Bluetooth to the receiving gadget or, after focusing, with an optical cable to the spectrometer and computer.

### TEM Spectroscopy for Synthesized AgNP in MIP Membrane Structure.

Transmission electron microscopy (TEM) was carried out to study the morphology and size distribution of AgNPs in the MIP membranes. Since the reduction of silver ions was used to synthesize AgNPs, the preparation of samples for TEM imaging was performed using the same protocol of polymerization, except the MIP membrane was prepared on a copper grid. The monomer mixture containing 10 mg ethyl-2-oxocyclopentanecarboxylate, 9 mg acrylamide, 162 mg TGDMA; 29 mg OUA, 100 μl DMF, 30 mg polyethyleneglycol Mr 20,000, 0.2 mg 2,2′-dimethoxy-2-phenylacetone and 1.5 mM AgNO₃ was used for the polymerization. The polymer was formed on the surface of copper grid 3 mm diameter. The copper grid was placed on glass slides (13×25.4 mm size) surface. Then 0.5 μl of the monomer mixture was dropped onto the grid and another glass slide was placed over the sample. The monomers mixtures were polymerized between two glass slides which were kept with two binder clips (18 mm size). The polymerization was initiated using UV light (2=365 nm, intensity 3.4 Wm⁻²) and performed for 30 min. Finally, the MIPs synthesized on the copper grid surface were used for TEM spectroscopy investigations. Transmission electron microscope (JEM-1230, JEOL, Japan) with an accelerating voltage 50-120 kV was used for the investigation of AgNP formed in the MIP membrane structure.

### Example 3—Device Construction and Testing

We present the development of novel nanostructured composite MIP membranes with embedded in situ-synthesized Ag nanoparticles that were used as recognition elements for a portable sensor device prototype for the ultrasensitive MIP-assisted detection of aflatoxin B1 due to the phenomenon of plasmon-enhanced fluorescence (PEF). The AFB1-sensitive sensor chips were produced through immobilization of thin MIP membranes on the surface of glass slides. A method of covalent immobilization of thin MIP films capable of aflatoxin B1 recognition was developed. Acrylamide is one functional monomer capable of effective recognition of aflatoxin B1 through selective binding. [Sergeyeva, T., et al., (2019) Talanta, 201, 204-210.] The covalent immobilization procedure for the acrylamide-based MIP thin film on the surface of the glass slides based on application of γ-methacryloxypropyltrimethoxysilane was developed and optimized.

The registration principle of the proposed sensor based on MIP-AgNP membranes is based on selective binding of aflatoxin B1 present in the analyzed samples by nanoreceptor sites and the measurement of plasmon-enhanced fluorescence signal of aflatoxin molecules excited by UV irradiation. The fluorescent sensor signals were registered by the portable fluorimeter constructed for this purpose. It was shown that formation of silver nanoparticles in the MIP membranes' structure provide amplification of the fluorescence signal due to the increased local electromagnetic field intensity resulting from localized surface plasmon resonance excitation in nanoparticles.

The developed sensor chips based on immobilized acrylamide-containing MIP thin films were produced and tested for their ability to generate fluorescent sensor response as a result of AFB1 binding. Blank membranes were obtained using the same compositions of monomers except for the dummy template (ethyl-2-oxocyclopentanecarboxylate) that was added to the MIP compositions only. The thickness of the MIP membranes immobilized on glass surfaces comprised 80±10 μm. The aflatoxin B1 detection is possible due to its natural ability to fluorescence. The sensor signals were generated by UV-irradiation (λ=365 nm nm) after incubation of the developed sensor chips in 15-500 ng mL⁻¹ solutions of AFB1, while blue fluorescence of the toxin of interest selectively adsorbed on the surface of the chips was registered at 2=420 nm directly at the sensor chip surface with both the standard laboratory spectrofluorimeter and the portable fluorimeter specially constructed for the purpose of AFB1 in-field detection.

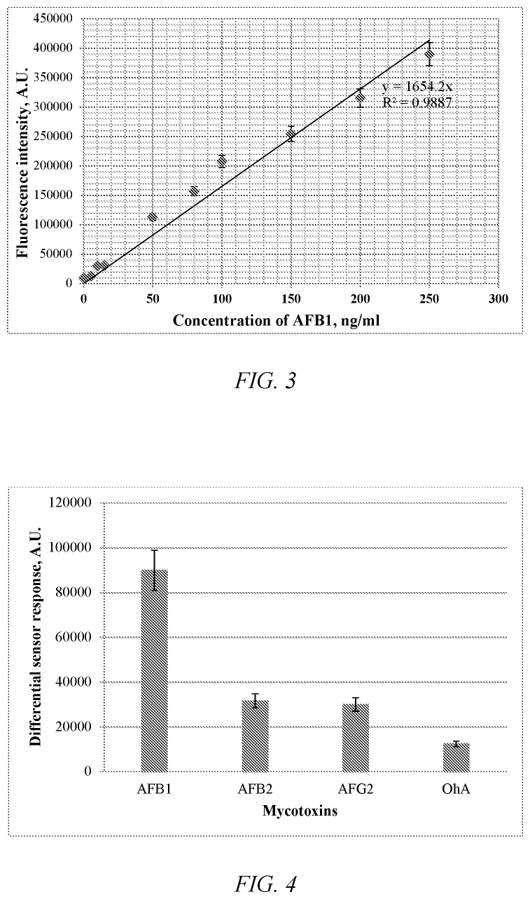

Typical calibration plots for the fluorescent sensor systems based on acrylamide-containing MIP membranes immobilized on the surface of glass chips are presented in FIG. 2. The minimal limit for AFB1 detection was estimated as 10 ng/ml, while linear dynamic range comprised 10-250 ng/ml (FIG. 3). Storage stability for proposed sensing chips for AFB1 fluorescent detection was assessed during 6 months storage at room temperature.

As compared to the previously-reported approach³⁹ based on application of 60 μm-thick free-standing MIP membranes the developed sensor chips provided some improvement of the LOD (10 ng mL⁻¹ as compared to 15 ng mL⁻¹) as well as better reproducibility. Moreover, the proposed sensor chips are also characterized by the superior mechanical stability.

The ability of the developed fluorescent sensors based on MIP-based sensor chips to discriminate between the toxin of interest and its potential interferents that are often present in the extracts of the analyzed cereal samples was investigated using the other structurally similar fluorescent mycotoxins, i.e. aflatoxins B2 and G2 (AFB2, AFG2) as well as ochratoxin A (OhA). The differential fluorescent sensor signals, generated with the immobilized MIP-based sensor chips in response to addition 100 ng mL⁻¹ AFB1, AFB2, AFG2 and OhA, were evaluated (FIG. 4).

It was shown that the MIP membranes immobilized on the glass surfaces demonstrated predominant binding of the toxin of interest as compared to the potentially interfering fluorescent substances (FIG. 4), however, the achieved LOD for AFB1 detection (10 ng mL⁻¹) required further improvement to ensure sensitive detection of the target toxin in food and feeding stuffs. Therefore, the initial MIP composition was further modified through incorporating Ag nanoparticles in their structure.

We demonstrated in-situ formation of Ag nanoparticles directly in the MIP thin film structure during the procedure of polymer formation. Silver nanoparticles (AgNPs) were obtained during the reduction of silver nitrate (AgNO₃) added to the initial composition for the imprinted polymer synthesis in appropriate concentration to the metallic silver. The proposed synthetic route is based on formation of silver nanoparticles in MIP thin film structure due to AgNO₃ reduction during the pre-heating step of the initial monomer mixture used for the MIP formation followed by UV-initiated photopolymerization. To the best of our knowledge, proposed approach of Ag nanoparticles synthesis in the MIP structure for the effective fluorescent detection of aflatoxins was used for the first time. Silver nitrate was added to the initial monomer mixture to the final concentrations 0.6-2.9 mM.

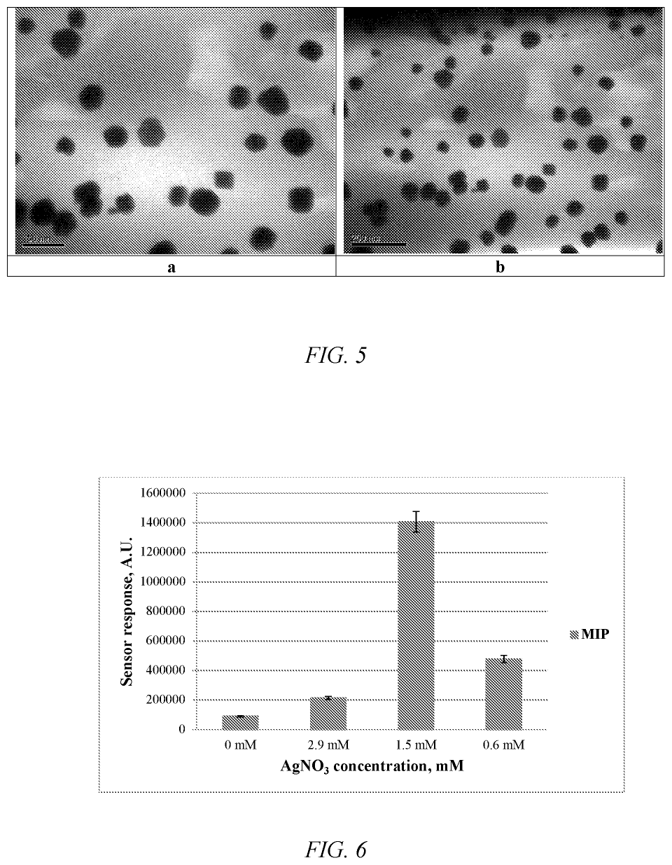

The morphology of the AgNPs formed in the structure of the AFB1-selective MIP films was investigated using TEM method. FIG. 5 shows TEM images of the MIP thin films obtained by the method of in situ polymerization with the Ag NPs embedded in the polymer structure. As one can see, the shape of AgNPs formed in the structure of the MIP membranes at the optimized conditions (from the monomer compositions containing 1.5 mM AgNO₃ in the initial mixture of monomers for the MIP synthesis) was close to spherical shape with the size of 30-70 nm. The nanoparticles were evenly distributed in the structure of the polymer. According to modeling of the system with spherical silver nanoparticle, this size provided some level of fluorescence enhancement, and, at the same time, the possibility remained for further improving of the already reached enhancement results by increasing the nanoparticle size. From the other side, some deviations from spherical shape (i.e. rectangular, hexagonal) were also observed on the TEM image. This limits the results of the used model as well as the possibility to further growing of size for spherical nanoparticles under the used conditions of polymerization. Besides, the presence of copper grid during polymerization procedure, that could influence the reduction of silver ions and, consequently, the morphology of synthesized nanostructures, should be taken into consideration.

All the MIP and blank Ag-containing polymers immobilized on the surface of glass slides were used as sensor chips for aflatoxin B1 detection. The ability of AgNPs formed in the polymer structure to decrease minimal detection limit of the target mycotoxin due to the plasmon-enhanced fluorescence phenomenon was investigated. All synthesized membranes were tested as for their ability to bind AFB1 from aqueous solutions as well as to generate the fluorescent sensor response (FIG. 6). For the MIP membranes synthesized from the mixtures containing 1.5 mM AgNO₃ a 14-fold increase in the value of the fluorescent sensor responses as compared to the AgNP-free MIP membranes as a result of AFB1 addition was observed (FIG. 6). Both the higher and lower AgNO₃ concentrations added to the MIP composition resulted in significantly lower (2.1-4.5-fold) enhancement effects.

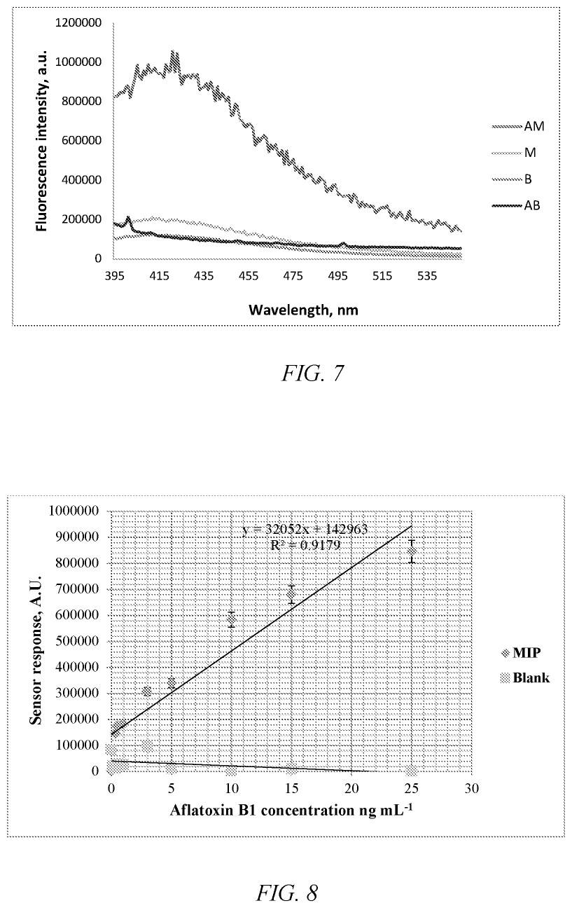

The ability of AgNPs formed in the polymer structure to increase intensity of natural fluorescence of aflatoxin B1 due to the plasmon-enhanced fluorescence phenomenon is shown in FIG. 7. Addition of AgNO₃ to the initial composition of monomers leads to further formation of Ag nanoparticles in the polymer structure and results not only in a significant increase in values of the sensor responses, but also in a significant increase of the imprinting factors of the aflatoxin B1-selective MIP membranes. We observed a 4.7-fold increase in the imprinting factor at 100 ng mL⁻¹ AFB1 concentration.

The AgNP-containing MIP-based sensor chips were tested as selective elements of the fluorescent sensor and the ability of AgNPs to improve minimal detection limit of aflatoxin B1 detection was studied. Typical calibration plots for the fluorescent sensor systems based on acrylamide-containing MIP-membranes synthesized with addition 1.5 mM AgNO₃ are presented in FIG. 8. It was shown that the minimal detection limit of aflatoxin B1-selective sensor system was decreased 33-fold due to the effect of plasmon-enhanced fluorescence: the LOD was decreased down to 0.3 ng mL⁻¹ for AgNPs-based MIP membranes as compared to 10 ng mL⁻¹ for the unmodified MIP-based sensor systems. The linear dynamic range of the fluorescent sensor comprised 0.3-25 ng mL⁻¹ (FIG. 8).

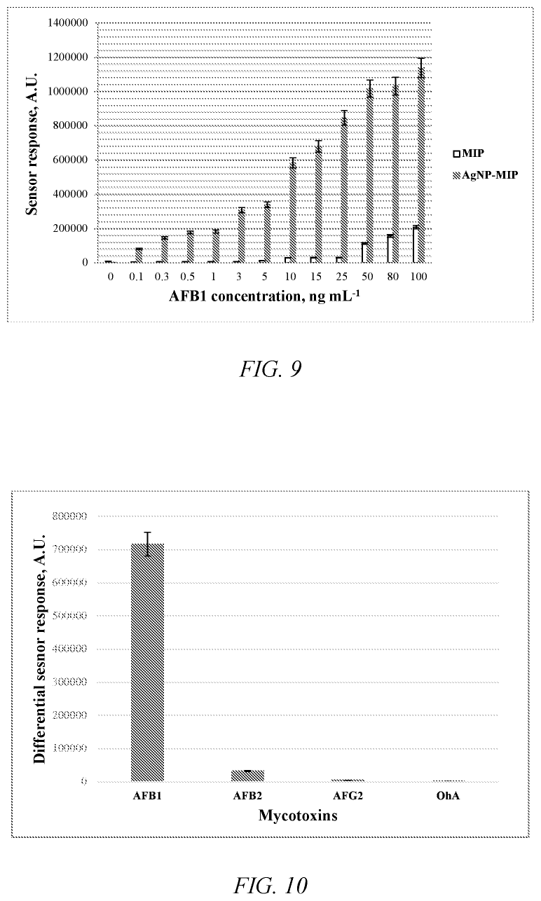

It was also demonstrated that the values of fluorescent sensor responses of the sensors based on AgNPs-containing MIP membranes were 40-80% higher as compared to those of AgNPs-free MIPs (FIG. 9). It was clearly shown that AgNPs-containing MIP thin films demonstrate superior properties as sensitive elements of a portable fluorescent sensor system as compared to unmodified MIPs. They are capable of generation of significantly higher sensor responses, which resulted in the 33-fold decrease of the minimal detection limit for AFB1.

The selectivity of the newly synthesized AgNP-containing sensor chips was also evaluated (FIG. 10). The differential fluorescent sensor signals, generated with the AgNPs-containing MIP sensor chips in response to addition of 15 ng mL⁻¹ AFB1, AFB2, AFG2 and OTA, were evaluated.

The developed sensor system based on AgNPs-containing MIP sensor chips was characterized with superior selectivity. The sensor responses initiated by the addition of the potentially interfering AFB1 structural analogues (AFB2, AFG2, and OhA) were negligible as compared to those generated by the target mycotoxin (AFB1). Moreover, the selectivity of the AgNPs-containing MIP-based sensors was much better as compared to the selectivity of the unmodified MIP membranes shown in FIG. 4.

In order to evaluate the performance of the developed portable fluorescent sensor system based on AgNPs-containing MIP sensor chips for the purposes of the food quality monitoring, the real cereal samples were tested as for AFB1 contamination using the developed method. Five different maize flour samples were tested. The first three samples (sample No. 1, 2 and 3) were naturally contaminated with AFB1 and provided by Romer Labs (Kiev, Ukraine): sample No. 1—“Romer Labs CheckSample Survey Aflatoxins in corn (CSSMY013-M17411A)”; sample No. 2—“Quality control material Aflatoxins in corn, low level”, sample No. 3—“Quality control material Aflatoxins in corn, mid-level”. All the naturally contaminated samples were characterized by the manufacturer as for the AFB1 content by standard analytical methods (HPLC and ELISA). Also AFB1-free maize flour samples produced by two different manufacturers (“Lavka Tradytsiy”, Ukraine (sample No. 4), “DobrodiyaFoods”, Kiev, Ukraine (sample No. 5)) and purchased in the local supermarket were used in the investigation. The procedure of AFB1 extraction from maize flour was made using 80:20 v/v acetonitrile: H₂O solution according to the standard procedure as described [Sergeyeva, T., et al., (2019) Talanta, 201, 204-210.]. The AFB1-free flour extracts were spiked with 1 (sample No. 4) and 5 (sample No. 5) ng mL⁻¹ AFB1. Results of aflatoxin B1 detection in real maize flour samples using the fluorescent sensor system based on AgNPs-containing AFB1-selective MIP thin films are presented in Table 2, below.

The results of the test are presented in Table 2. The results demonstrate the efficacy of the disclosed AgNPs-containing MIP-based sensor system for the monitoring AFB1 contamination of the cereal samples. The obtained results of analytical identification of aflatoxins corresponded to the results obtained by the traditional instrumental and immunochemical methods.

In summary, a portable fluorescent sensor system based on AgNP-containing MIP membranes capable of selective aflatoxin B1 recognition suitable for in-field application is disclosed herein. AgNPs were synthesized in situ directly in the structure of the MIP membranes during polymerization procedure. This approach resulted in a 33-fold decrease in the detection limit as well as in a significant increase in the overall value of the sensor responses due to LSPR phenomenon as compared to the sensors based on AgNP-free MIP sensor chips. The minimal detection limit for AFB1 was estimated as 0.3 ng mL⁻¹, the linear dynamic range of the developed fluorescent sensor was 0.3-25 ng mL⁻¹. Negligible binding of interferents that can be potentially present in analyzed cereal extracts was observed by the aflatoxin B1-selective AgNPs-containing MIP sensor chips. Formation of 25-65 nm diameter AgNPs in the structure of the MIP membranes evenly distributed in the polymer was confirmed by TEM studies. The developed sensor system was effective for the aflatoxin B1 analysis in both spiked and naturally contaminated samples of cereal extracts.

### Glossary of Terminology

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

Other than in the operating examples, or unless otherwise expressly specified, all of the numerical ranges, amounts, values and percentages such as those for amounts of materials, times and temperatures of reaction, ratios of amounts, values for molecular weight (whether number average molecular weight (“Mₙ”) or weight average molecular weight (“M_{w}”), and others in the following portion of the specification may be read as if prefaced by the word “about” even though the term “about” may not expressly appear with the value, amount or range. Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used.

As used herein, the term “composition” is intended to encompass a product comprising the specified ingredients in the specified amounts, as well as any product which results, directly or indirectly, from combination of the specified ingredients in the specified amounts.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

As used herein, the term “sample” refers to a small quantity of matter or product that is representative of a larger population under investigation.

As used herein, the phrases “conditions sufficient for” or “conditions effective to” refer to any environment that permits the desired activity, for example, that permit specific binding or hybridization between the toxins in a sample and an MIP.

As used herein, the term “contact” refers to placement in direct physical association and includes both in solid and liquid form. For example, contacting can occur between an MIP and a biological sample in solution where the biological sample is being tested for the detection of an aflatoxin.

As used herein, the term “detect” and/or “detection” refers to the act of, or steps associated with, determining if an agent (such as a toxin, biomolecule, amino acid, nucleic acid molecule, and/or organism) is present or absent in a sample being screened. In some examples, this can further include quantification. For example, use of the disclosed methods permit detection of aflatoxins in a sample.

As used herein, the term “support” or “solid support” refers to a material which is insoluble, or can be made insoluble by a subsequent reaction, that can serve as a mount for an MIP or thin film. A support for an MIP or other thin film according to the invention will generally be free of fluorescence upon incidence of UV light in the absence to the target analyte. One advantageous solid support is a glass slide.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. All references to the function log default to e as the base (natural log) unless stated otherwise (such as log₁₀).

A fluorophore (or fluorochrome, similarly to a chromophore) is a fluorescent chemical compound that can re-emit light upon light excitation. Aflatoxins, such as aflatoxin b1, are known fluorophores. Other fluorophores that can be used in contemplated detections systems according to the invention include a variety of mycotoxins (aflatoxins—AFB1, AFB2, AFG1, AFG2, deoxynivalenol DON, zearalenone ZEA, fumonisins FB1, FB2, and FB3), ochratoxin OhA, trichothecene. In addition to the general class of mycotoxins (aflatoxins, etc.), one can also detect fluorescent pathogens in humans and animals such as bacterial infections and fungi.

Aptamers are oligonucleotide sequences with a length of about 25-100 bases that have binding properties and specificities mimicking monoclonal antibodies. [Shuaijian Ni, et al., ACS Applied Materials & Interfaces 2021 13 (8), 9500-9519.] Peptide aptamers have also been described. Aptamers generally fold into diverse three-dimensional structures that bind to specific targets, such as toxins and antigens. An aptamers selection process, named Systematic Evolution of Ligands by Exponential Enrichment (SELEX), was developed in 1990 by Tuerk and Gold, [Tuerk, C.; Gold, L. Systematic Evolution of Ligands by Exponential Enrichment: Rna Ligands to Bacteriophage T4 DNA Polymerase. Science 1990, 249, 505-510, DOI: 10.1126/science.2200121] and Ellington and Szostak. [Ellington, A. D.; Szostak, J. W. In Vitro Selection of Rna Molecules That Bind Specific Ligands. Nature 1990, 346, 818-822, DOI: 10.1038/346818a0]. Aptamers have already been generated demonstrating selective binding of a multitude of targets, such as small organic molecules, nucleic acids, amino acids, antibiotics, peptides, proteins, bacteria, viruses, or even whole living cells. [Menger M, Yarman A, Erdőssy J, Yildiz H B, Gyurcsányi R E, Scheller F W. MIPs and Aptamers for Recognition of Proteins in Biomimetic Sensing. Biosensors (Basel). 2016; 6(3):35.]

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

All references cited in the present application are incorporated in their entirety herein by reference to the extent not inconsistent herewith.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

TABLE 1
Compositions of the monomer mixture for the MIP and blank
membranes' synthesis

| Monomer/oroligomer | MIP | | Blank | |
|---|---|---|---|---|
| Ethyl-2-oxocyclopentanecarboxylate | 10 | mg | — | |
| Acrylamide | 9 | mg | 9 | mg |
| TGDMA | 162 | mg | 162 | mg |
| OUA | 29 | mg | 29 | mg |
| DMF | 100 | μl | 100 | μl |
| polyethyleneglycol (Mw 20 000) | 30 | mg | 30 | mg |
| 2,2′-dimethoxy-2-phenylacetone | 0.2 | mg | 0.2 | mg |

TABLE 2
Detection of AFB1 in maize samples by the portable fluorescent
AgNPs-containing MIP-based sensor system and traditional
analytical methods.

| | | Amount of AFB1 in the |
|---|---|---|
| | Amount of AFB1 in the | sample (according to portable |
| | sample (according to | fluorescent MIP-based sensor |
| Sample No. | traditional method) | system) |
| 1 | 7 μg kg⁻¹ | 6.4 ± 3.2 μg kg⁻¹ |
| 2 | 4.2 ± 1.7 μg kg⁻¹ | 4.5 ± 2 μg kg⁻¹ |
| 3 | 7.3 ± 2.9 μg kg⁻¹ | 7.7 ± 0.9 μg kg⁻¹ |
| 4 | 1 ng mL⁻¹ | 1.9 ± 0.9 ng mL⁻¹ |
| | | (0.8 μg kg⁻¹) |
| 5 | 5 ng mL⁻¹ | 5.4 ± 0.8 ng mL⁻¹ |
| | | (2 μg kg⁻¹) |

1. A sensor chip comprising a MIP membrane or thin film having embedded silver nanoparticles (AgNP), wherein the MIP or thin film exhibits a binding affinity for a fluorophore of interest whereby the AgNP in the MIP enhances the fluorescence of the fluorophore upon application of UV irradiation.

2. The sensor chip according to claim 1 wherein the MIP selectively binds a fluorophore selected from the group consisting of aflatoxin B1 (AFB1), aflatoxin B2 (AFB2), aflatoxin G1 (AFG1), aflatoxin G2 (AFG2), deoxynivalenol (DON), zearalenone (ZEA), fumonisin B1 (FB1), fumonisin B2 (FB2), fumonisin B3 (FB3), ochratoxin (OhA), and trichothecene.

3. The sensor chip according to claim 1 wherein the fluorophore of interest is a mycotoxin.

4. The sensor chip according to claim 1 wherein the fluorophore of interest is an aflatoxin.

5. The sensor chip according to claim 4 wherein aflatoxin is selected from the group consisting of aflatoxin B₁, aflatoxin B₂, aflatoxin G₁, and aflatoxin G₂.

6. The sensor chip according to claim 1 wherein the AgNP are roughly spherical in shape and have a size of about 30-70 nm.

7. The sensor chip according to claim 1 wherein the AgNP are nanoparticles are evenly distributed in the structure of the MIP membrane.

8. The sensor chip according to claim 1 wherein the MIP comprises a polymer selected form the group consisting of acrylamide (AA), poly(ethyleneimine), poly(hydroxyethyl methacrylate), poly(vinylpyrrolidone), novolak, poly(4-vinylphenol), poly(4-vinylphenol)-co-(methyl methacrylate), and poly(styrene-co-allyl alcohol).

9. The sensor chip according to claim 1 wherein the MIP comprises an aptamer that selectively binds a fluorophore of interest.

10. The sensor chip according to claim 9 wherein the aptamer selectively binds an aflatoxin.

11. A sensor chip comprising a MIP membrane or thin film having embedded nanoparticles selected from the group consisting of silver nanoparticles, gold nanoparticles, copper nanoparticles and aluminum nanoparticles, wherein the MIP or thin film exhibits a binding affinity for a fluorophore of interest whereby the nanoparticle in the MIP enhances the fluorescence of the fluorophore upon application of UV irradiation.

12. A method of detecting a fluorophore in a sample comprising the steps of:
providing a sensor chip according to claim 1;
contacting the sensor chip with a sample to be tested for the presence of a fluorophore under conditions effective to cause binding between fluorophores in the sample and the sensor chip;
irradiating the contacted sensor chip with UV light; and
detecting the resulting fluorescence in the sample, whereby the resulting fluorescence is enhanced by the AgNP nanoparticle in the sensor chip, wherein if the detected fluorescence exceeds a threshold level the fluorophore is present in the sample.

13. The method according to claim 12 wherein the fluorophore of interest is an aflatoxin.

14. The method according to claim 12 wherein the nanoparticle is AgNP.

15. The method of detecting a fluorophore in a sample according to claim 12 further comprising the step of quantifying the concentration of fluorophore in the sample by comparing the detected fluorescence in the sample with a standard curve of fluorescence for the concentration of the fluorophore.

16. The method of detecting a fluorophore in a sample according to claim 12 wherein the excitation wavelength is about 365-400 nm and the measurement range is about 395-550 nm.

17. The method of detecting a fluorophore in a sample according to claim 12 wherein the excitation wavelength is about 365 nm.

18. An optical system for a portable fluorimeter comprising two collimators, a bandpass filter, a sample holder, sensor chip according to claim 2, and a longpass filter.

19. A method for the synthesis of AgNP-containing MIP membranes from monomer mixtures comprising the steps of providing a solution containing methacrylate monomers or oligomers and AgNO₃ in dimethylformamide, forming the AgNP in the structure of the polymeric membranes during the pre-heating step and polymerzing the solution using UV-initiated polymerization.

20. The method for the synthesis of AgNP-containing MIP membranes from monomer mixtures according to claim 19 wherein the concentration of AgNO3 is between 1.0 and 2.0 mM.

21. (canceled)

22. (canceled)

23. (canceled)
